(19) United States
(12) Patent Application Publication
(10) Pub. No.: US 2020/0152309 A1
(43) Pub. Date: May 14, 2020

(54) MEDICAMENT DISPENSING DEVICE

(71) Applicant: DROPP HEALTH LTD, London (GB)

(72) Inventors: Kaveh MEMARI, London (GB); Adrian Bennett, Orpington (GB)

(21) Appl. No.: 16/682,934

(22) Filed: Nov. 13, 2019

(30) Foreign Application Priority Data
Nov. 13, 2018 (GB) 1818472.1

(51) Int. Cl. G16H 20/13; G06N 20/00

(52) U.S. Cl. CPC G16H 20/13; G06N 20/00

(57) ABSTRACT

The invention provides for a metered dose delivery device adapted to dispense a metered dose of a medicament, wherein the metering of the dose is controlled based on a user input and user specific data. The device includes a user interface adapted to receive a user input. The device further includes a communication interface adapted to communicate with a processing unit and adapted to transmit user specific data to, and receive user specific data from, the processing unit. The transmitted and received user specific data includes the user input. The device further includes a control unit adapted to control the metering of the dose of the medicament based on the user input and the received user specific data.

### FIELD OF THE INVENTION

This invention relates to the field of medicament dispensing devices, and more specifically to the field of devices for dispensing a metered dose of a medicament.

### BACKGROUND OF THE INVENTION

There are a number of conditions for which a user may be required to regularly self-medicate. Certain medicaments employ a dispensing mechanism for delivering the medicament from a container to the user.

Typically, the dispensing mechanism is adapted to dispense a predetermined amount of the contained medicament. The predetermined amount (i.e. dose) is usually fixed at the point of manufacture. However, some devices are known which employ a dispensing mechanism that can dispense different amounts (dosages) of medicament. In other words, such devices may control the metering of the dose of medicament.

### SUMMARY OF THE INVENTION

The invention is defined by the claims.

According to examples in accordance with an aspect of the invention, there is provided a metered dose delivery device adapted to dispense a metered dose of a medicament, the device comprising:

a user interface, wherein the user interface is adapted to receive a user input;

a communication interface adapted to communicate with a processing unit, wherein the communication interface is adapted to:
transmit user specific data to the processing unit, wherein the user specific data comprises the user input;
receive user specific data from the processing unit; and

a control unit, wherein the control unit is adapted to control the metering of the dose of the medicament based on the user input and the received user specific data.

The device provides for a medicament delivery device, wherein the metering of the dose is controlled based on a user input and user specific data received from a processing unit.

As the metering of the dose of the medicament is controlled based on a user input and the received user data, the dosage may be controlled according to the needs of a specific user. Thus, the controlling of the dosage may be performed in an accurate manner that is tailored to the needs of an individual. In this way, the efficacy of a given medicament may be maximised for each user.

The device transmits user specific data to the processing unit, for example periodically, to ensure the received user specific data is kept up to date. The user interface may be any suitable user interface.

The device provides for a means of acquiring any user specific data and/or feedback on the efficacy of the medicament in a robust fashion without requiring significant additional hardware or effort on the part of the user. Thus, the device eliminates the requirement to separately collect data from the user, which may introduce potential errors into the data.

Accordingly, the risk of collecting erroneous data from the user, which increased with the time taken to acquire the data, may be reduced, or eliminated.

In an embodiment, the processing unit is a remote processing unit. In this way, the user specific data may be processed away from the device. For example, the remote processing unit may be a smartphone in communication (wired or wireless) with the device. Alternatively, the device may connect to an internet access point, in which case the remote processing unit may be a cloud server. Further, the device may transfer the data to a smartphone, which in turn transfers the data to a server to be processed.

In an embodiment, the processing unit is adapted to analyse the transmitted user specific data, thereby generating analysis data and wherein the received user specific data comprises the analysis data. In this way, the received user data may contain further information to be accounted for determining the metering of the dose.

In a further embodiment, the processing unit is adapted to perform the analysis by way of a machine learning algorithm. In this way, the analysis of the user data may adapt over time.

In an arrangement, the user specific data comprises a user profile. In this way, the user specific data may be represented in a profile of their preferences and results. This may be compared to other users to compare the relative efficacy of one metering device to another.

In a further arrangement, the user profile comprises historic user information. Historic user information may be used to further adjust the metering of the dose to best suit an individual user.

In an embodiment, the user input comprises user feedback. In this way, the user may provide a subjective input thereby personalising further metering decisions for the dose delivery.

In a further embodiment, the user feedback comprises a perceived efficacy of the dispensed metered dose of the medicament. This data may be used to identify an effective result for a given patient, which may then be repeated in future.

In an embodiment, the user interface comprises a user identification unit. In this way, it is possible to ensure only the appropriate user may use the device (e.g. fingerprint scanner).

In an embodiment, the user interface is further adapted to provide a prompt signal to the user. In this way, the device may inform a user when a dose should be dispensed, for example when consistent timing is required.

In a further embodiment, the prompt signal comprises:

a visual signal;

an audible signal;

a tactile signal; and

an electronic signal.

A variety of signals may be employed depending on user preference and/or need. Further, the electronic signal may be used to communicate with a companion device such as a smartphone.

In an arrangement, the device comprises a medicament identification unit adapted to identify the medicament and wherein the user specific information comprises the identity of the medicament. In this way, the medicament being dispensed may be accounted for in the user specific data. This may be used where more than one medicament is dispensed by the same device. Further, this may be used to prevent the device from dispensing an incorrect medicament.

In an embodiment, the medicament comprises a mixture of a plurality of preliminary medicaments. In this way, the medicaments may be mixed in the device.

In a further embodiment, a composition of the mixture is based on the received user specific data. The composition of the mixture may change depending on the specific user data.

According to examples in accordance with an aspect of the invention, there is provided a method for dispensing a metered dose of a medicament, the method comprising:

receiving a user input;

transmitting user specific data to a processing unit;

receiving user specific data from the processing unit;

controlling the metering of the dose of medicament based on the user input and the received user specific data; and

dispensing the metered dose of medicament.

### BRIEF DESCRIPTION OF THE DRAWINGS

Examples of the invention will now be described in detail with reference to the accompanying drawings, in which:

FIG. 1 shows an example of a medicament dispensing device according to an aspect of the invention;

FIG. 2 shows a schematic representation of the device of FIG. 1 having an internal processing unit;

FIG. 3 shows a schematic representation of the device of FIG. 1 having a remote processing unit;

FIG. 4 shows a further example of a medicament dispensing device according to an aspect of the invention; and

FIG. 5 illustrates an example of a processing system.

### DETAILED DESCRIPTION OF THE EMBODIMENTS

It should be understood that the Figures are merely schematic and are not drawn to scale. It should also be understood that the same reference numerals are used throughout the Figures to indicate the same or similar parts.

In the context of the present application, where embodiments of the present invention constitute a method, it should be understood that such a method is a process for execution by a computer, i.e. is a computer-implementable method. The various steps of the method therefore reflect various parts of a computer program, e.g. various parts of one or more algorithms.

Also, in the context of the present application, a (processing) system may be a single device or a collection of distributed devices that are adapted to execute one or more embodiments of the methods of the present invention. For instance, a system may be a personal computer (PC), a server or a collection of PCs and/or servers connected via a network such as a local area network, the Internet and so on to cooperatively execute at least one embodiment of the methods of the present invention.

The invention provides for a metered dose delivery device adapted to dispense a metered dose of a medicament, wherein the metering of the dose is controlled based on a user input and user specific data. The device includes a user interface adapted to receive a user input. The device further includes a communication interface adapted to communicate with a processing unit and adapted to transmit user specific data to, and receive user specific data from, the processing unit. The transmitted and received user specific data includes the user input. The device further includes a control unit adapted to control the metering of the dose of the medicament based on the user input and the received user specific data.

In other words, there is provided a device for dispensing a controlled dose of a contained medicament. The device is adapted to receive a user input, which forms part of user specific data that is transmitted from the device to a processing unit. The metering of the dose is controlled based on user specific data received from the processing unit and the user input.

Put another way, a device is provided for dispensing a metered dose of a medicament, which can be in communication with a processing unit. The device receives a user input that forms at least part of a set of user specific data that is transmitted to the processing unit by the device. The metering of the dose is then controlled by user specific data received from the processing unit.

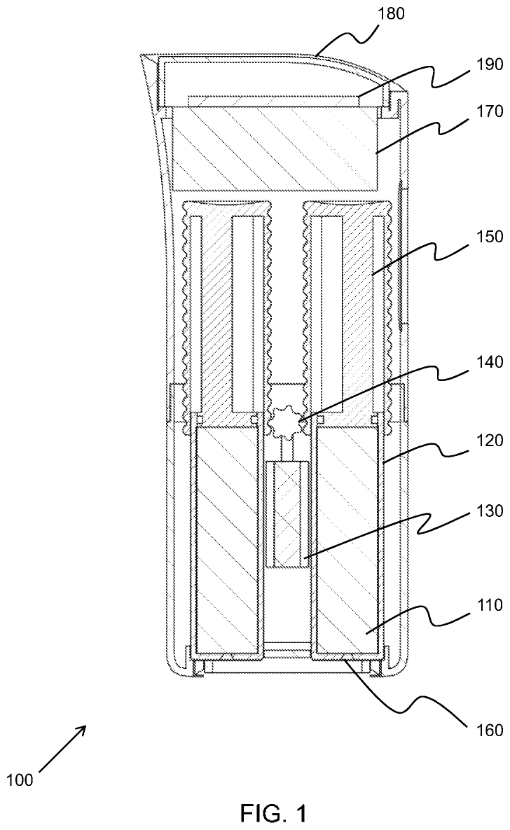

FIG. 1 shows a schematic representation of an example of a metered dose delivery device 100 according to an aspect of the invention. The general operation of such a delivery device will first be described with respect to FIG. 1.

The metered dose delivery device 100 holds a medicament 110 in a holding chamber 120. Upon the activation of a motor 130, a driving cog 140 engages with a piston 150 and causes the piston to descend into the holding chamber.

As the piston descends into the holding chamber, the medicament will be displaced and begin leaving the holding chamber by way of an exit valve 160. The distance travelled by the piston will dictate the amount of the medicament that leaves the holding chamber.

In other words, the metering of the dosage of medicament dispensed by the device may be controlled by altering the distance moved by the piston.

In this way, the device may dispense the medicament in an unheated and chemically unchanged form. Thus, temperature sensitive medicaments may be dispensed from the device with a reduced, or eliminated, risk of undergoing chemical decomposition during delivery.

Alternatively, the device may comprise a mechanical, or electronic, pump for dispensing the medicament. Further, the device may be adapted to include an atomizer, thereby providing for the dispensing of the medicament in the form of a spray.

In addition, the device comprises a battery 170 for powering the motor 130.

Further, there is provided a user interface 180 adapted to receive a user input from a user of the device. The user interface may be any means for receiving a user input. By way of example, the user interface may include one or more of: a button; a dial; a slider; a lever; a light sensor; a touch sensor; and a touchscreen.

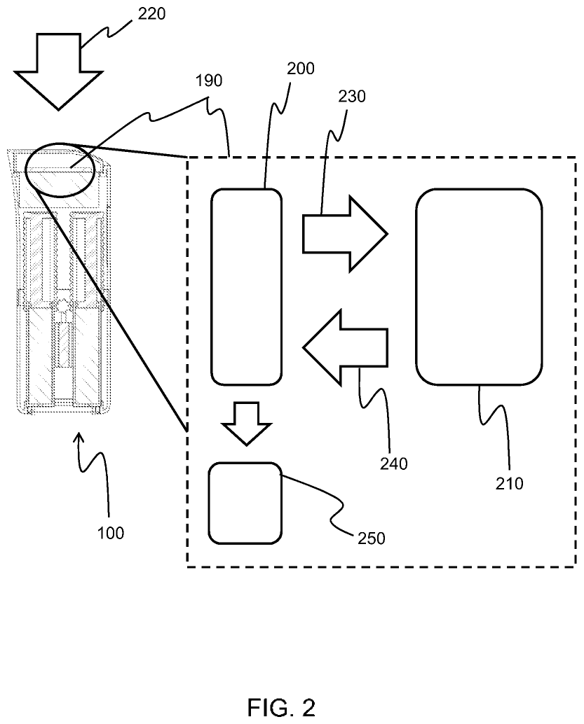

In the example shown in FIG. 1, the user interface 180 is a button which may be depressed by the user to provide the user input. Upon receiving the user input, the motor 130 may be activated and a dose of the medicament 110 dispensed. Further examples of possible user inputs and user interfaces are discussed below with reference to FIG. 2.

The device further contains a processing system 190 in communication with the user interface 180, the motor 130 and the battery 170. The processing system is described further below with reference to FIG. 2.

The device may also include an internal temperature sensor, which activates an electronic warming mechanism when required to warm the medicament within the holding chamber, or a separate delivery chamber, to achieve a desired temperature ahead of delivery.

FIG. 2 shows a schematic representation of the device 100 of FIG. 1, wherein the processing system 190 includes a communication interface 200 and a processing unit 210.

The device receives a user input 220 at the user interface. By way of example, the user presses a button on the device to dispense the medicament. Subsequently, the communication interface 200 transmits 230 user specific data to the processing unit, the user specific data at least partially including the user input.

It should be noted that the communication unit may transmit user specific data to the processing unit at any time, for example on a predetermined schedule.

The processing unit may then process the user specific data transmitted from the communication unit. Following the processing of the user specific data, the communication interface receives 240 the user specific data from the processing unit.

This data may then be sent to a control unit 250, which may then control the metering of the dose of the medicament based on the data. The controlling of the metering may be performed before the current dose is dispensed or after the current dose is dispensed, meaning that the metering will be controlled based on this data for the subsequent dispensing of the medicament.

Accordingly it is possible for the device to internally perform the entire process of obtaining, processing and acting on user specific data.

The user input may include user feedback, which may include, for example, a user's perceived efficacy of the dispensed medicament. The user interface may comprise multiple user input means. For example, there may be a button to dispense the medicament in addition to a dial, which may be used to indicate the efficacy of the dispensed medicament by turning the dial.

The user feedback may then be provided to the processing system 210 as part of the transmitted user specific data, which is ultimately used to control the metering of the dose dispensed by the device. For example, user feedback indicating that the dispensed dose of medicament was effective may result in the control unit maintaining the metering of the dose at the current level; whereas, user feedback indicating that the dispensed dose was not effective may result in the control unit altering the metering of the dose for the next dispensing. The user interface may be further adapted to receive more detailed user feedback, for example indicating that the dispensed dose was too little or too much.

The user interface 180 may be adapted to include a user identification unit, in which case the user input may include user identification data. For example, the user identification unit may include: an alphanumerical input, wherein the user input may comprise a passcode; a fingerprint scanner, wherein the user input may include a fingerprint; an iris scanner, wherein the user input may include an iris scan; and a voice recognition unit, wherein the user input includes an audible component.

The user identification unit may be included to ensure that only an authorised user of a device may be able to dispense the medicament. In particular, the user identification unit may prevent the unauthorised dispensing of a prescription medicament.

The user interface may be further adapted to provide a prompt signal to the user. The prompt signal may be any signal encouraging the user to interact with this device. For example, the user interface may provide a prompt signal to remind the user to dispense a dose of the medicament at regular intervals or to provide feedback on a recently dispensed dose. The prompt signal may be any suitable signal type, such as: a visual signal, such as a flashing LED or text where the user interface includes an LCD display; an audible signal, such as a beep or a verbal prompt; a tactile signal, such as a vibration; and an electronic signal adapted to communicate with another device to provide a suitable prompt to a user, such as a smartphone.

The processing unit may be adapted to process the transmitted user specific data in a number of ways. For example, the processing unit may perform various forms of analysis on the transmitted user data, thereby generating analytical data that may be supplied to the control unit for better controlling the metering of the dose. Said analysis may be performed by way of a machine learning algorithm, which may be used to better identify patterns and trends in the user specific data.

The user specific data may include a user profile, which may contain a wide array of user information (such as height, age and weight) that may be used to accurately determine a required dose of a given medicament, thereby increasing the accuracy of the metering of the dose by the device. The user profile may also include historic user information, such as: personal medical history; family medical history; genetic information; and the like. The historic user information may also include dosage history that can be viewed and exported for review by care takers, doctors or other medical personnel.

In the case that the user input, and so the user specific information, includes user feedback, the user feedback may be used to establish a medicament efficacy profile over time for particular medicaments and concentrations for a given user. Further, the processing unit may capture and amalgamate large data sets to identify patterns of optimal treatment for various conditions over time. This data may be used for a single patient, or may be combined with data from a plurality of patients having similar profiles, conditions or medicaments.

The processing unit may be further adapted to generate recommendations based on: a condition and/or symptoms of the user, based on standard procedures associated with said condition and/or symptoms; comparable conditions of other users, for example based on the dosage data of users having a higher efficacy, based on their feedback, for the user group; and the user profile including details such as age, weight, condition, other medications being consumed, lifestyle factors and genetic profile that may affect efficacy.

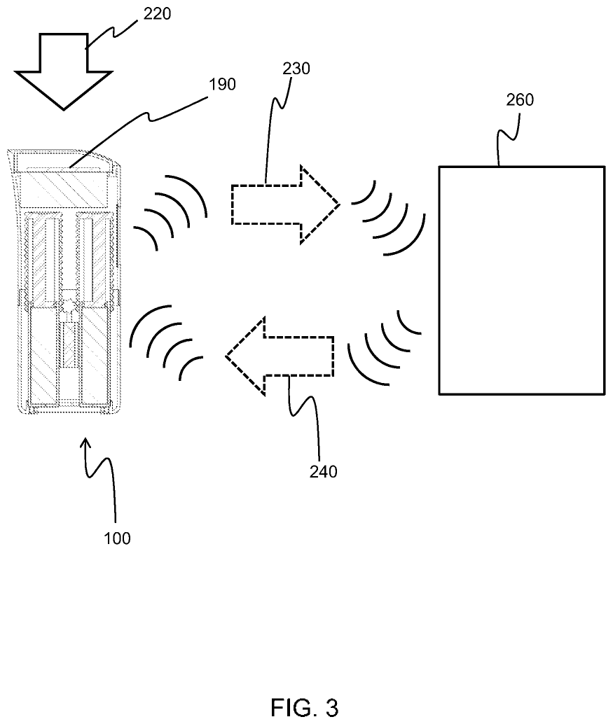

FIG. 3 shows a schematic representation of the device 100 of FIG. 1, wherein the processing system 190 includes a communication interface in communication with a remote processing unit 260.

In this example, the communication interface is adapted to transfer/receive the user specific data to/from the remote processing unit 260. The remote processing unit may be adapted to perform any of the processing functions detailed above with reference to FIG. 2. Further, the analysis of the user specific data may be performed on a combination of a processing unit located within the device and a remote processing unit.

The remote processing unit may be any processor that is not located within the device 100 and capable of communicating with the communication interface of the processing system 190. The remote processing unit may communicate with the communication interface by way of a wired connection or a wireless connection.

For example, the device may connect wirelessly, for example via a Bluetooth or Wi-Fi connection, to a smartphone of the user. The smartphone may then act as the remote processing unit for handling the user specific data. Further, the user may access the data on the smartphone by way of an associated app.

Alternatively, the smartphone may simply act as a conduit transferring the user specific data to a server, which may then act as the remote processing unit 260. In this case, the user may, for example, access the data by way of a smartphone app, a desktop app or a web app.

In other words, the device 100 may be connected to a smart device of the user, such as a smartphone or smartwatch, for capturing and displaying the user specific data via a wired or wireless connection.

The connected, or associated, device may then provide a dashboard for viewing the user specific data, such as dosage data and a current and/or historic user profile. The displayed user specific data may further include past medication and relevant user specific data relating to underlying symptoms or conditions.

In the example of a remote processing unit, the user specific data received by the device 100 from the remote processing unit 260 may include an additional user input. For example, the additional user input may include external user identification by establishing a connection with an authorised remote processing unit, such as an authorised smartphone.

The device 100 may connect the user directly to a medical professional by way of a connected smart device, which may also act as the remote processing unit 260. Further, the device may send and receive alerts based on the user specific data to appointed recipients or third parties approved by the user.

More specifically, the user may elect to share part or all of the user specific data with any third-party user, such as a medical healthcare provider. The user specific data may share different subsets of information with different third parties. For example, a user may share all of the user specific data with a doctor but only share data indicating the remaining contents of a holding chamber of the device with a prescription company.

In other words, the user specific data may inform the circle of care of the user, which may include care givers, medical professionals and experts, about dosage, efficacy of dispensed medicaments over time and how the user is self-reporting their experiences of their condition.

The user specific data may be anonymised and aggregated with user specific data relating to other users according to similar data values, such as dosage history and condition, to in order to recognise population trends. Thus, the user specific data may be used to help maximise the efficacy of treatment for a variety of symptoms. The individual user will benefit in seeing the collective data, which may highlight treatments that other users have found effective for a given condition.

Further, the user specific data may be supplemented by situational data captured through additional user devices that capture health data, activity data or related user data to provide additional context on the user and conditions around dispensed dosages over time.

The user specific data may be analysed and used as a discovery mechanism for other supporting lifestyle changes for users to support their quality of life, such as support group recommendations, dietary recommendations, exercise recommendations and the like. Further the user specific data may be used to discover and expand the user's support network, for example, by recommending medical professionals.

The user specific data may be used to control the metering of the dispensed dose to account for the build-up of tolerance to the medicament by the user, i.e. the increased usage of a medicine with smaller effect, over time. The analysis of the user specific data may be used to make a recommendation to mitigate the development of a tolerance.

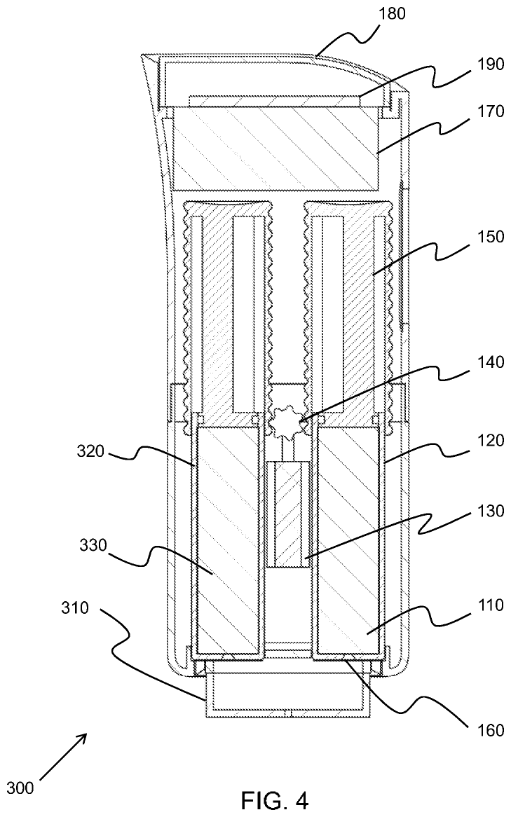

FIG. 4 shows an embodiment of a device 300 for dispensing a metered dose of a medicament.

In addition to the features described above with reference to FIG. 1, the device 300 further comprises a mixing chamber 310 in communication with the holding chamber 120 and an additional holding chamber 320. The additional holding chamber may hold the same medicament 110 as holding chamber 120; however, in this example, the additional holding chamber contains a different medicament 330. In other words, the device may hold multiple different medicaments for dispensing to a user. Further, each medicament may be subject to different metering criteria according to the user specific data.

In the case where the medicaments are different, both medicaments may be dispensed into the mixing chamber 310, where they are mixed before being dispensed to the user. The composition of the mixture may be based on the user specific data.

In other words, the device may generate various dosages using multiple holding chambers in communication with the mixing chamber. For example, two different medicaments may be mixed at different concentrations at different points of the day.

The device may include a medicament identification unit, which is adapted to identify the medicament held in a holding chamber. In this case, the identifications of the medicaments may form part of the user specific data transferred to the processing unit.

By way of example, each medicament may be uniquely identifiable via an electronic tag placed on a removable holding chamber that is read by the medicament identification unit when the holding chamber is placed into the device. This may ensure precise control of the dispensing of the contents of the holding chambers.

Further, the holding chambers containing the medicaments may be made to be tamper resistant and may only deliver their contents when placed within the device and activated by a user of device.

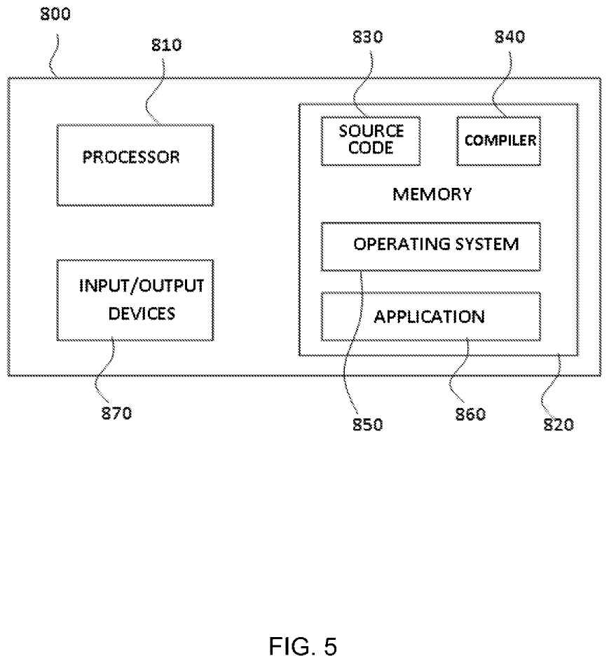

FIG. 5 illustrates an example of a processing system 800 within which one or more parts of an embodiment may be employed. Various operations discussed above may utilize the capabilities of the processing system 800. For example, one or more parts of the processing system 190 housed within the device 100, 300 may be incorporated in any element, module, application, and/or component discussed herein.

Generally, in terms of hardware architecture, the processing system 800 may include one or more processors 810, memory 820, and one or more I/O devices 870 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components. For example, the I/O devices and the local interface may make up at least part of the communication interface 200 discussed above.

The processor 810 is a hardware device for executing software that can be stored in the memory 820. The processor 810 can be virtually any custom made or commercially available processor, a central processing unit (CPU), a digital signal processor (DSP), or an auxiliary processor among several processors associated with the processing system 800, and the processor 810 may be a semiconductor based microprocessor (in the form of a microchip) or a microprocessor.

The memory 820 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM) or the like, etc.). Moreover, the memory 820 may incorporate electronic, magnetic, optical, and/or other types of storage media. Note that the memory 820 can have a distributed architecture, where various components are situated remote from one another, but can be accessed by the processor 810.

The software in the memory 820 may include one or more separate programs, each of which comprises an ordered listing of executable instructions for implementing logical functions. The software in the memory 820 includes a suitable operating system (O/S) 850, compiler 840, source code 830, and one or more applications 860 in accordance with exemplary embodiments. As illustrated, the application 860 comprises numerous functional components for implementing the features and operations of the exemplary embodiments. The application 860 of the processing system 800 may represent various applications, computational units, logic, functional units, processes, operations, virtual entities, and/or modules in accordance with exemplary embodiments, but the application 860 is not meant to be a limitation.

The operating system 850 controls the execution of other computer programs, and provides scheduling, input-output control, file and data management, memory management, and communication control and related services. It is contemplated by the inventors that the application 860 for implementing exemplary embodiments may be applicable on all commercially available operating systems.

Application 860 may be a source program, executable program (object code), script, or any other entity comprising a set of instructions to be performed. When a source program, then the program is usually translated via a compiler (such as the compiler 840), assembler, interpreter, or the like, which may or may not be included within the memory 820, so as to operate properly in connection with the O/S 850. The I/O devices 870 may include various input devices. Furthermore, the I/O devices 870 may also include various output devices, for example but not limited to a display, etc. Finally, the I/O devices 870 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 870 also include components for communicating over various networks, such as the Internet or intranet.

If the processing system 800 is a PC, workstation, intelligent device or the like, the software in the memory 820 may further include a basic input output system (BIOS) (omitted for simplicity). The BIOS is a set of essential software routines that initialize and test hardware at startup, start the O/S 850, and support the transfer of data among the hardware devices. The BIOS is stored in some type of read-only-memory, such as ROM, PROM, EPROM, EEPROM or the like, so that the BIOS can be executed when the processing system 800 is activated.

When the processing system 800 is in operation, the processor 810 is configured to execute software stored within the memory 820, to communicate data to and from the memory 820, and to generally control operations of the processing system 800 pursuant to the software. The application 860 and the O/S 850 are read, in whole or in part, by the processor 810, perhaps buffered within the processor 810, and then executed.

When the application 860 is implemented in software it should be noted that the application 860 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The application 860 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

In the context of the present application, where embodiments of the present invention constitute a method, it should be understood that such a method is a process for execution by a computer, i.e. is a computer-implementable method. The various steps of the method therefore reflect various parts of a computer program, e.g. various parts of one or more algorithms.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a storage class memory (SCM), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A metered dose delivery device adapted to dispense a metered dose of a medicament, the device comprising:
a user interface, wherein the user interface is adapted to receive a user input;
a communication interface adapted to communicate with a processing unit, wherein the communication interface is adapted to:
transmit user specific data to the processing unit, wherein the user specific data comprises the user input;
receive user specific data from the processing unit; and
a control unit, wherein the control unit is adapted to control the metering of the dose of the medicament based on the user input and the received user specific data.

2. A device as claimed in claim 1, wherein the processing unit is a remote processing unit.

3. A device as claimed in claim 1, wherein the processing unit is adapted to analyse the transmitted user specific data, thereby generating analysis data and wherein the received user specific data comprises the analysis data.

4. A device as claimed in claim 3, wherein the processing unit is adapted to perform the analysis by way of a machine learning algorithm.

5. A device as claimed in claim 1, wherein the user specific data comprises a user profile.

6. A device as claimed in claim 6, wherein the user profile comprises historic user information.

7. A device as claimed in claim 1, wherein the user input comprises user feedback.

8. A device as claimed in claim 7, wherein the user feedback comprises a perceived efficacy of the dispensed metered dose of the medicament.

9. A device as claimed in claim 1, wherein the user interface comprises a user identification unit.

10. A device as claimed in claim 1, wherein the user interface is further adapted to provide a prompt signal to the user.

11. A device as claimed in claim 10, wherein the prompt signal comprises:
a visual signal;
an audible signal;
a tactile signal; and
an electronic signal.

12. A device as claimed in claim 1, wherein the device comprises a medicament identification unit adapted to identify the medicament and wherein the user specific information comprises the identity of the medicament.

13. A device as claimed in claim 1, wherein the medicament comprises a mixture of a plurality of preliminary medicaments.

14. A device as claimed in claim 13, wherein a composition of the mixture is based on the received user specific data.

15. A method for dispensing a metered dose of a medicament, the method comprising:
receiving a user input;
transmitting user specific data to a processing unit;
receiving user specific data from the processing unit;
controlling the metering of the dose of medicament based on the user input and the received user specific data; and
dispensing the metered dose of medicament.
